(19) United States
(12) Patent Application Publication
(10) Pub. No.: US 2009/0311799 A1
(43) Pub. Date: Dec. 17, 2009

(54) Nucleic Acid Materials for Nonradiative Energy Transfer and Methods of Production and Use

(76) Inventors: Gregory A. Sotzing, Storrs, CT (US); Jeffrey A. Stuart, Columbia, CT (US); Yogesh J. Ner, Willimantic, CT (US)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/484,509

(22) Filed: Jun. 15, 2009

Related U.S. Application Data
(60) Provisional application No. 61/061,459, filed on Jun. 13, 2008.
(60) Provisional application No. 61/144,028, filed on Jan. 12, 2009.

(51) Int. Cl. G01N 21/00; C07H 21/00; B29C 47/00; B29C 39/00; G21G 5/00

(52) U.S. Cl. 436/172; 536/22.1; 264/465; 264/299; 250/492.1

(57) ABSTRACT

Nucleic acid materials for FRET-based luminescence and methods of making and using the nucleic acid materials are provided. The nucleic acid materials provide an innovative and synergistic combination of three disparate elements: a nucleic acid material, the processing technique for forming a nucleic acid material into films, fibers, nanofibers, or non-woven meshes, and nonradiative energy transfer. This combination can be formed into electrospun fibers, nanofibers, and non-woven meshes of a nucleic acid material-cationic lipid complex with encapsulated chromophores capable of nonradiative energy transfer such as efficient Förster Resonance Energy Transfer (FRET).

### CROSS REFERENCE TO RELATED APPLICATIONS

This application claims the benefit of U.S. Provisional Application No. 61/061,459, filed Jun. 13, 2008 and U.S. Provisional Application No. 61/144,028, filed Jan. 12, 2009, both of which are hereby incorporated by reference in their entireties.

### STATEMENT REGARDING FEDERALLY SPONSORED RESEARCH

This application and research leading to this application were funded in part by National Science Foundation grants CHE 0349121 and DMR 0502928. Accordingly, the U.S. Federal Government may have certain rights in this application.

### FIELD

This application relates to the field of optoelectronics and more particularly relates to materials for nonradiative energy transfer.

### BACKGROUND

Förster Resonance Energy Transfer (FRET) is a mechanism of nonradiative energy transfer between two molecules, a donor and an acceptor. When the donor molecule is in its excited state, it can transfer energy by a nonradiative, long range dipole-dipole mechanism to the acceptor molecule. The efficiency of nonradiative energy transfer depends on factors such as the distance between the donor and acceptor molecules, the relative orientation of the dipole moments of the donor emission and the acceptor absorption, and the spectral overlap of the donor emission spectrum and the acceptor absorption spectrum. A key challenge is obtaining the appropriate spatial organization for efficient energy transfer. To achieve this organization, a structural matrix is required that furnishes both proper orientation and appropriate proximity between the donor and acceptor molecules.

Nucleic acids are materials that can form complexes with a wide variety of molecules through intercalation, groove-binding, and ionic interactions. Because of the intrinsic lattice structure of nucleic acids, guest molecules are isolated and have defined spatial orientations. Nucleic acids can also complex with ionic surfactants or with lipids with ionic head groups. Nucleic acids are natural materials and renewable resources that are both biocompatible and biodegradable. Nonradiative energy transfer has been studied for nucleic acid-lipid complexes in solution; however, to date, there have been no reports of nonradiative energy transfer in solid state nucleic acid materials.

Currently, white light is produced in both compact fluorescent lights (CFLs) and white light emitting diodes by excitation of phosphor coatings doped with rare earth metals. The quality of the white light is a function of the composition of the phosphor coating. Disposal of these units poses an environmental risk due to the mercury in CFLs, and the rare earth metals in both the CFLs and white light LEDs (light-emitting diodes).

Accordingly, it is an object of the present invention to provide a material that efficiently produces visible or near infrared luminescence with minimal or no environmental risk.

It is a further object of the present invention to provide a material that efficiently produces white light with minimal or no environmental risk.

It is another object of the present invention to provide a device containing a nucleic acid based material that is capable of nonradiative energy transfer.

It is another object of the present invention to provide a process to detect and quantify an analyte where the analyte causes a change in nonradiative energy transfer that produces light emission and the process measures the change in light emission caused by the analyte.

### SUMMARY

Described herein are nucleic acid materials for nonradiative energy transfer, particularly FRET-based luminescence, methods of making and using the materials, and devices containing the materials. The materials utilize an innovative and synergistic combination of three disparate elements: a nucleic acid material; a processing technique for forming a nucleic acid material into films, fibers, nanofibers, or non-woven meshes; and nonradiative energy transfer. Nanofibers are fibers with a diameter of between approximately 2 nm and approximately 5 μm. More preferably nanofibers have a diameter of between about 30 nm and about 500 nm. In one embodiment, the nucleic acid, processing technique, and nonradiative energy transfer combination results in electrospun nanofibers and non-woven meshes of a nucleic acid-cationic lipid complex that acts as a host matrix for FRET.

Nucleic acids have unique abilities to interact with a variety of molecules through multiple mechanisms. These interactions lead to materials with well-defined nanoscale morphologies that are suitable for a variety of applications. Nucleic acids impose a defined spatial organization and orientation on the small molecules with which they interact and simultaneously prevent aggregation of these molecules.

In one embodiment a nucleic acid material having a plurality of donor and acceptor molecules incorporated therein is provided. These donor and acceptor molecules are capable of a nonradiative energy transfer, such as FRET. These donor and acceptor molecules may be dye molecules or chromophores. These donor and acceptor molecules have a 3-dimensional organization fixed by the nucleic acid material. The plurality of donor and/or acceptor molecules optionally contain at least two acceptor molecules that emit at different wavelengths. Alternatively, the plurality of donor and/or acceptor molecules contain at least three different molecules and at least one of the three molecules functions as both a donor and an acceptor.

A preferred nucleic acid is deoxyribonucleic acid (DNA). Another preferred nucleic acid is double-stranded ribonucleic acid (RNA).

The nucleic acid may be in the form of a nucleic acid molecule complexed with an ionic surfactant or with a lipid with an ionic head group to improve processability. The preferred surfactant is a cationic surfactant. The preferred lipid is a lipid with a cationic head group. These nucleic acid materials are soluble in organic solvents and can be processed into thin films (e.g. by dip casting or spin casting) or into fibers, nanofibers, or non-woven meshes (e.g. by electrospinning) using techniques known to those skilled in the art. The processed complexes exhibit excellent thermal stability and transparency. Nucleic acid-surfactant complexes are also known to form a regular arrangement of alternate layers of nucleic acid and surfactant through nucleic acid self-assembly. The coordination between a nucleic acid and a surfactant results in a lamellar structure of aligned parallel nucleic acid sandwiched between surfactant layers.

Accordingly, in an embodiment, the nucleic acid material is a nucleic acid-ionic surfactant or nucleic acid-lipid complex in a solid state.

Preferably, the material is in the form of a film, a fiber, a nanofiber, or a non-woven mesh. Preferred embodiments are produced by electrospinning.

Other systems, methods, processes, devices, features, and advantages associated with the nucleic acid materials described herein will be or will become apparent to one with skill in the art upon examination of the following drawings and detailed description. All such additional systems, methods, processes, devices, features, and advantages are intended to be included within this description, and are intended to be included within the scope of the present invention.

### BRIEF DESCRIPTION OF THE SEVERAL VIEWS OF THE DRAWINGS

FIG. 1 is a schematic of cetyl trimethylammonium (CTMA) chloride complexed with DNA.

FIG. 2 is a 2-dimensional representation of DNA self assembly.

FIG. 3 is a schematic showing the lamellar structure of DNA and a cationic surfactant.

FIG. 4 is an X-ray diffraction pattern of a self-standing electrospun DNA-CTMA nanofiber mesh.

FIG. 5 is a graph showing normalized emission and UV-visible absorption spectra of nanofibers of DNA-CTMA-Cm102 (donor) and DNA-CTMA-Hemi22 (acceptor), respectively.

FIGS. 6A-B are fluorescence microscopy images of electrospun nanofibers of DNA-CTMA-donor (6A) and DNA-CTMA-multiple dye with acceptor:donor molar ratio 1:5 (6B).

FIG. 7 is a series of quenching curves for multi-dye doped DNA-CTMA nanofibers with varying ratios of acceptor to donor chromophores.

FIG. 8 is a graph showing FRET efficiency plotted against acceptor to donor ratio.

FIG. 9 is a color map for emission of DNA-CTMA nanofibers with varying acceptor to donor ratios.

FIG. 10 is a digital photograph of a commercially available LED, emitting at 400 nm, without (left) and with (right) FRET-based DNA nanofiber coating.

FIGS. 11A-B are graphs showing the comparative photostability of DNA and PMMA films prepared with equivalent amounts of Hemi 22.

FIG. 12 is a graph showing a photoluminance spectra of donor and acceptor channels formed in DNA-CTMA films.

### DETAILED DESCRIPTION

Nucleic acid materials for nonradiative energy transfer, particularly FRET-based luminescence, methods of making and using the materials, and devices containing the materials are provided herein. The materials efficiently produce white light or near infrared luminescence, are biodegradable and biocompatible, and pose little or no environmental risk.

The materials provided herein contain a nucleic acid material and multiple donor and acceptor molecules, which are embedded therein or associated therewith. The nucleic acid material described herein may further include an ionic surfactant or a lipid with an ionic head group. The preferred ionic surfactant is a cationic surfactant. The preferred lipid is a lipid with a cationic head group. The nucleic acid molecules may interact with the surfactant or lipid in the nucleic acid material to form a nucleic acid-surfactant complex or a nucleic acid-lipid complex. Preferably, the donor and acceptor molecules are donor-acceptor pairs capable of FRET. The materials are in a solid state and preferably in the form of a film, fiber, nanofiber, or non-woven mesh. Preferred embodiments are produced by dip casting, spin casting, or electrospinning. The device vice is covered with a thin layer of the material for nonradiative energy transfer. The nucleic acid materials described herein enable high dye loading, enhanced energy transfer between donors and acceptors due to their relative orientation and organization in the nucleic acid material, and increased photostability over conventional polymeric materials, such as polymethyl methacrylate (PMMA) and polyvinyl alcohol (PVA).

### Definitions

As used herein, the term “nucleic acid” refers to DNA, RNA and derivatives thereof, including, but not limited to, cDNA, gDNA, msDNA and mtDNA, mRNA, hnRNA, tRNA, rRNA, aRNA, gRNA, miRNA, ncRNA, piRNA, shRNA, siRNA, snRNA, snoRNA, stRNA, ta-siRNA, and tmRNA, as well as artificial nucleic acids including, but not limited to, peptide nucleic acid (PNA), glycol nucleic acid (GNA), threose nucleic acid (TNA), Morpholino and locked nucleic acid (LNA).

As used herein, the term “dye” refers to a coloring agent that tends to be organic in nature and is soluble.

A chromophore is the part of the dye molecule (i.e. the group of atoms) responsible for the electronic transition or absorption that gives the dye color. As used herein, the term “chromophore” refers to the group of atoms within a dye molecule that is responsible for the electronic transition and/or the dye molecule itself. A chromophore that emits light through fluorescence is a fluorophore.

### Nucleic Acids

Nucleic acids can form complexes with a wide variety of molecules through intercalation, groove binding, and ionic interactions. Because of the intrinsic lattice structure of nucleic acids, guest molecules are isolated and have defined spatial orientations. Nucleic acids can also complex with ionic surfactants and with lipids with ionic head groups. Nucleic acids are natural materials and renewable resources that are both biocompatible and biodegradable.

The nucleic acid structure allows simultaneous encapsulation of multiple donor and acceptor molecules by multiple mechanisms and imposes a defined spatial organization and orientation on those small molecules. Such an arrangement is required for efficient nonradiative energy transfer to occur. This increased level of organization over conventional polymers such as PMMA and PVA enables a high donor/acceptor molecule loading of up to 50%. The defined and constricted spatial positioning of the donor and acceptor molecules within the nucleic acid matrix also enhances the photostabilities of the donor and acceptor molecules.

A preferred nucleic acid material for use in the material provided herein is DNA. DNA is a natural material and a renewable resource. DNA has unique chemical and materials properties including the ability to interact with a wide variety of small molecules through multiple mechanisms such as intercalation, groove binding, and ionic interactions. Another preferred nucleic acid material is double-stranded RNA, which has similar abilities to interact with small molecules.

### Nucleic Acid Material Including Surfactant or Lipid

Aqueous nucleic acid solutions can be difficult to process in their native form due to strong intermolecular interaction and interwinding. Moreover, nucleic acids are not soluble in organic solvents. To overcome these problems, the nucleic acid used herein may be complexed with an ionic surfactant or a lipid with an ionic head group to improve processability. These complexes are soluble in organic solvents and can easily be processed into thin films (e.g. by dip casting or spin casting) or into fibers, nanofibers, or non-woven meshes (e.g. by electrospinning). The processed complexes have excellent thermal stability and transparency. Nucleic acid-surfactant complexes are also known to form a regular arrangement of alternate layers of nucleic acid and surfactant through nucleic acid self-assembly.

The preferred ionic surfactant is a cationic surfactant. The preferred lipid is a lipid with a cationic head group. Exemplary cationic surfactants are cationic quaternary ammonium cations or salts and include, but are not limited to, cetyl trimethylammonium (CTMA) chloride (also referred to as hexadecyl trimethylammonium chloride), cetylpyridinium chloride (CPC), polyethoxylated tallow amine (POEA), benzalkonium chloride (BAC), benzethonium (BZT) chloride, dioleoyl phosphatidylethanolamine (DOPE), cetyl trimethylammonium (CTAB), dioleoyltrimethylammonium propane (DOTAP), and dioctadecyldimethylammonium bromide (DODAB).

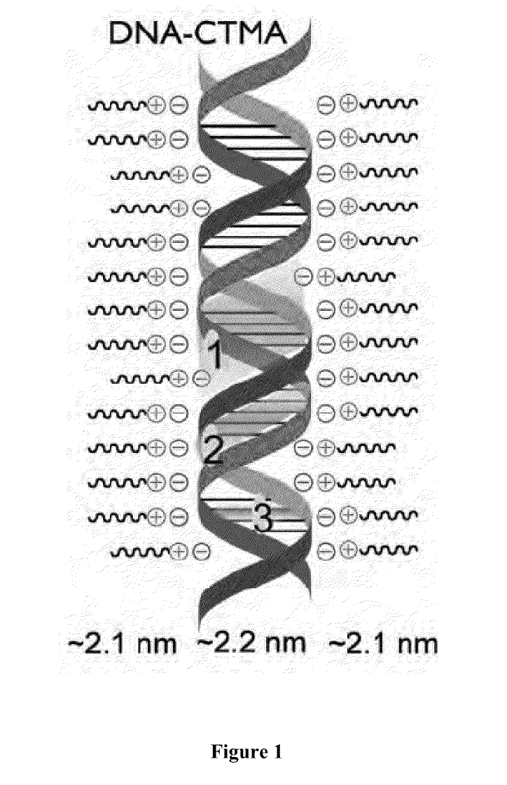

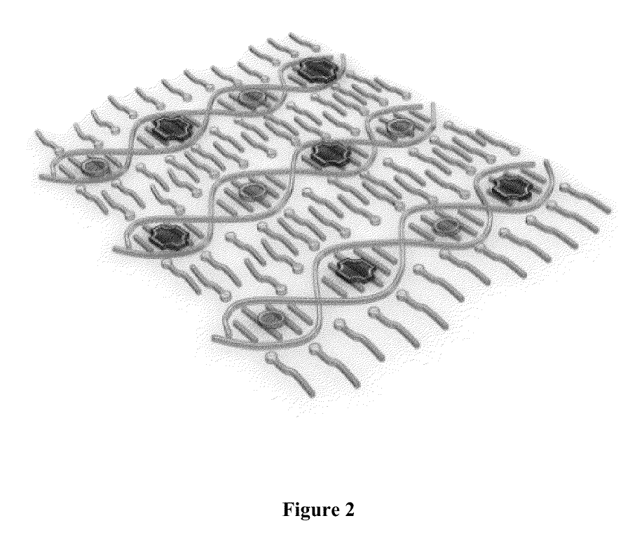

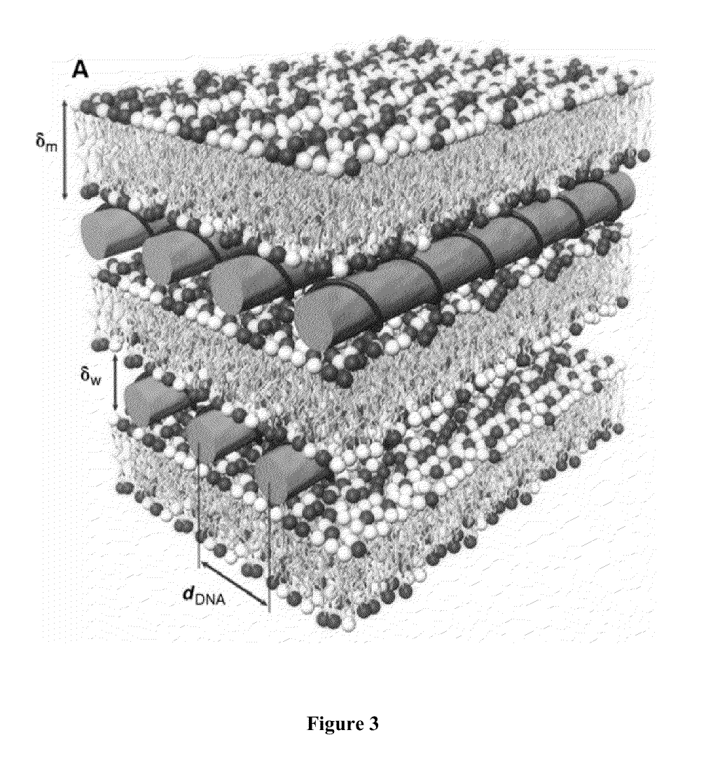

The coordination between a nucleic acid and a surfactant can result in a lamellar structure of aligned parallel nucleic acid sandwiched between surfactant layers. As an example, this coordination is shown in FIGS. 1-3 for DNA-CTMA. FIG. 1 is a schematic showing cationic CTMA complexed with DNA. (Radler, J. O., et al., Science 1997, 275(5301), 810-14.) Distances shown in FIG. 1 are (1) major groove, (2) minor groove, and (3) distance between ladder units. FIG. 2 is a schematic showing a 2D representation of DNA self assembly. FIG. 3 is a schematic showing the lamellar structure of DNA (rods) and the cationic surfactant DOPE. (Yu, Z., et al. Appl. Opt., 2007, 46(9): p. 1507-13).

As an example, in one embodiment surfactant-modified nucleic acid is prepared by slow stoichiometric addition of the cationic surfactant CTMA chloride to a nucleic acid in an aqueous concentration of 1% w/w to produce a nucleic acid-CTMA complex. The resulting precipitate can then be filtered, cleaned, and dried in accordance with methods well known to those skilled in the art.

The nucleic acid material containing surfactant described herein, and also referred to as the nucleic acid-surfactant complex, has advantageous properties that make it suitable for a variety of applications. The cationic surfactant or lipid that complexes with the DNA has a cationic head and a long alkyl chain tail. The tails of these molecules can be designed to carry functional groups including but not limited to chromophores and other active functional groups. Additionally, cationic surfactants are known to be antimicrobial and antifungal, thus the material of the invention also serves the purpose of an antimicrobial/antifungal material. Furthermore, nucleic acid-lipid complexes are highly optically transparent (up to 99%) and have very low background fluorescence, so they are suitable for optical applications. Thus, the novel properties of nucleic acid-lipid complexes can be exploited for fabrication of functional materials, including sensors and light sources.

In one embodiment, the nucleic acid material described herein can be used to detect the presence of an analyte. As a non-limiting example, an analyte may interact with a nucleic acid material provided herein through competitive binding. An interaction between an analyte and the nucleic acid material can change the emission characteristics of the chromophores in the nucleic acid material. This change in emission characteristics can be observed visually, e.g. as a color change, or spectroscopically.

In another embodiment two or more nucleic acid materials provided herein may be combined into a composition that is in the form of a film, fiber, nanofiber, or nonwoven mesh. Each of the nucleic acid materials independently provides nonradiative energy transfer that produces visible or near infrared luminescence. The combination of nucleic acid materials produces a luminescence that appears to have a single wavelength, e.g. appears to be a single color. By adjusting the amounts of each nucleic acid material in the composition the wavelength of the apparent luminescence can be tuned.

The nucleic acid materials described herein provide ample opportunities for small molecule interaction, either with the nucleic acid or with the surfactant or lipid component. Small molecules can associate with the nucleic acid material in a variety of ways including intercalation, groove-binding, and through ionic interactions. Multiple structural phases of the nucleic acid material provide a variety of specific nano-environments that can sequester small molecules. For example, the polar nucleic acid phase provides both ionic and dispersive bonding opportunities, while the surfactant or lipidic phase accommodates non-polar and hydrophobic molecules. The implication for nonradiative energy transfer technologies is that populations of donor and acceptor dyes can be isolated from one another within the same matrix, thereby allowing higher loading levels than are possible with other matrix materials. For example, DNA complexes can accommodate donor and acceptor molecules without aggregation until all DNA grooves incorporate donor and acceptor molecules. Theoretically, loadings up to 30% by weight are possible depending upon the molecular weight of the donor and acceptor molecules used. This is an advantage over conventional polymers such as PMMA and PVA because those conventional polymers lack an organized internal structure and, therefore, cannot prevent embedded dye molecules from interacting at higher concentrations which ultimately results in fluorescence quenching.

The small molecules can associate with the nucleic acid before or after the nucleic acid-surfactant or lipid complex is formed. If the molecules associate with the nucleic acid-surfactant (or lipid) complex after it is formed, they may associate with the complex either before processing while the complex is in solution or after processing while the complex is in the form of a solid film or fiber. Thus, films and fibers formed from the nucleic acid-surfactant (or lipid) complexes can be used to absorb small molecules to remove those molecules from a medium such as air or a solvent. Nucleic acid-surfactant (or lipid) complexes have particular affinity for aromatic molecules including, but not limited to, the dyes disclosed herein. Examples of such aromatic molecules also include polycyclic aromatic hydrocarbons, a class of harmful chemicals present in automotive emission. These aromatic molecules are also carcinogens, so nucleic acid-lipid complexes have utility in detoxification applications.

A vast variety of molecules can interact with nucleic acids. A particular donor or acceptor molecule's solubility will determine the methods by which a homogeneous matrix of nucleic acid and that molecule may be produced. For example, if a donor or acceptor molecule is water soluble, the molecule may be added to an aqueous nucleic acid solution before the nucleic acid is complexed with a surfactant or lipid. If the donor or acceptor molecule is soluble in alcohol and/or chloroform, the molecule may be added to a solution of a nucleic acid-surfactant (or lipid) complex in alcohol or chloroform or a mixture thereof. If the donor or acceptor molecule is soluble in a solvent other than water, alcohol, or chloroform, a nucleic acid-surfactant (or lipid) complex may be processed into a preferred shape, e.g. film or fiber, and the processed nucleic acid-surfactant (or lipid) complex may then be dipped into a solution of donor or acceptor molecules to produce the donor/acceptor-nucleic acid-surfactant (or lipid) matrix. If the donor or acceptor molecule is soluble in multiple solvents, these methods can be used alternatively or simultaneously.

### Donor and Acceptor Molecules

Preferred small molecules for interacting with the nucleic acid material include donor and acceptor molecules, also referred to herein as donor and acceptor chromophores or dyes. The preferred donor and acceptor molecules are donors and acceptors capable of nonradiative energy transfer, such as FRET. FRET is dependent upon the spacing and relative orientation of the donor and acceptor molecules. FRET efficiency is related to, among other things, the concentration of the donor and acceptor molecules. At low concentrations FRET may not occur or will occur with low efficiency. At high concentrations, aggregation may inhibit or quench FRET. The unique properties of nucleic acids tend to sequester donor and acceptor molecules in such a way that their relative orientation and separation are locked in an arrangement that facilitates efficient energy transfer and allows higher loading of the donor or acceptor molecules without detrimental aggregation. This arrangement cannot be duplicated in an amorphous polymer matrix.

The structure of nucleic acids provides a convenient matrix for donor and acceptor molecules that positions the donor and acceptor molecules in a constant relative spatial arrangement. This arrangement fixes both the distance between the donor and acceptor molecules and the relative orientation of the donor and acceptor molecules, which enhances FRET and enhances luminosity by approximately two orders of magnitude as compared to more conventional (i.e. non-biological) polymeric matrices. Furthermore, donor and acceptor molecules associated with nucleic acids via intercalation or groove binding exhibit enhanced fluorescence due to reduced self-quenching through aggregation.

The interactions between nucleic acid-surfactant complexes and donor and acceptor molecules prevent the donor and acceptor molecules from forming aggregates in solid state films and fibers. In the solid state, the donor and acceptor molecules can associate with the nucleic acid-surfactant complex in various ways including intercalation, major/minor groove binding, and/or in between the surfactant molecules. The various possible conformations may explain the role of the nucleic acid in isolating individual donor and accept molecules and the observed fluorescence enhancement and amplified spontaneous emission in DNA-CTMA dye doped films. In addition to enhancement of these photophysical properties, such configurations lead to significant changes in the photochemical properties of the dyedoped films of nucleic acids. For example, isolation of donor and acceptor molecules in DNA can significantly prevent photodegradation due to dimerization. DNA is a strong UV absorber which can also act as a shield for a donor or acceptor molecule's photodegradation.

Donor and acceptor molecules suitable for use in the nucleic acid materials provided herein include any donor and acceptor molecules capable of FRET. For example, suitable donor and acceptor molecules include, but are not limited to, organic dyes and pigments, oligomeric compounds, and conducting polymers. For example, suitable donor and acceptor molecules include, but are not limited to rhodamines; fluoresceines; cyanines; porphyrins; naphthalimides; perylenes; quinacridons; benzene-based compounds such as distyrylbenzene (DSB) and diaminodistylrylbenzene (DADSB); naphthalene-based compounds such as naphthalene and Nile red; phenanthrene-based compounds such as phenanthrene; chrysene-based compounds such as chrysene and 6-nitrochrysene; perylene-based compounds such as perylene and N,N′-bis(2,5-di-t-butylphenyl)-3,4,9,10-perylene-di-carboxyl amide (BPPC); coronene-based compounds such as coronene; anthracene-based compounds such as anthracene and bisstyrylanthracene; pyrene-based compounds such as pyrene; pyran-based compounds such as 4-(di-cyanomethylene)-2-methyl-6-(para-dimethylaminostyryl)-4H-pyran (DCM); acridine-based compounds such as acridine; stilbene-based compounds such as stilbene; thiophene-based compounds such as 2,5-dibenzooxazolethiophene; benzooxazole-based compounds such as benzooxazole; benzoimidazole compounds such as benzoimidazole; benzothiazole-based compounds such as 2,2′-(para-phenylenedivinylene)-bisbenzothiazole; butadiene-based compounds such as bistyryl(1,4-diphenyl-1,3-butadiene) and tetraphenylbutadiene; naphthalimide-based compounds such as naphthalimide; coumarin-based compounds such as coumarin; perynone-based compounds such as perynone; oxadiazole-based compounds such as oxadiazole; aldazine-based compounds; cyclopentadiene-based compounds such as 1,2,3,4,5-pentaphenyl-1,3-cyclopentadiene (PPCP); quinacridone-based compounds such as quinacridone and quinacridone red; pyridine-based compounds such as pyrrolopyridine and thiadiazolopyridine; spiro compounds such as 2,2′,7,7′-tetraphenyl-9,9′-spirobifluorene; and metallic or non-metallic phthalocyanine-based compounds such as phthalocyanine (H₂Pc) and copper phthalocyanine.

The donor/acceptor molecules can also be from the various organometallic complexes such as 3-coordination iridium complex having on a ligand 2,2′-bipyridine-4,4′-dicarboxylic acid, factris(2-phenylpyridine)iridium (Ir(Ppy)₃), 8-hydroxyquinoline aluminum (Alq₃), tris(4-methyl-8-quinolinolate)aluminum (III) (Almq₃), 8-hydroxyquinoline zinc (Znq₂), (1,10-phenanthroline)-tris-(4,4,4-trifluoro-1-(2-thienyl)-butane-1,3-dionate), europium (III) (Eu(TTA)₃(phen)), 2,3,7,8,12,13,17,18-octaethyl-21H, and 23H-porphin platinum (II).

The choice of donor and acceptor molecules is important because intelligent selection of donor and acceptor molecules results in tunable color emission, including the ability to precisely control color temperature of white light emission. A molecule may function as either a FRET donor or a FRET acceptor depending on the molecule with which it is paired. Furthermore, three donor/acceptor molecules may be matched such that the first molecule acts as a donor for the second, the second molecule acts as an acceptor for the first molecule and as a donor to the third molecule, and the third molecule acts as an acceptor for the second molecule. For matched FRET donor and acceptor molecules the emission spectra of the donor chromophore overlaps with the absorption spectra of the acceptor chromophore. Emission can be tuned with selection of donors and acceptors and with selection of the relative ratio of donor and acceptor molecules.

One of the goals of the materials provided herein is to achieve a maximum number of color states in the visible region from simultaneous emission of the chromophores. To achieve that goal, the choice of donor is one with excitation wavelength in the long wavelength ultraviolet (UV) region (targeted 360-400 nm). Thus, donor molecules preferred for use in the nucleic acid materials described herein include but are not limited to chromophores selected from the following classes: coumarins, ATTO dyes, AlexaFluor dyes, Hoechst dyes, and pyrenes. Each of these classes of chromophores includes at least some chromophores that absorb in the ultraviolet spectrum. This absorption allows these chromophores to be used to generate white light from an emitting LED. Alternatively the material is coated onto a ultraviolet diode and absorbs in the range of a commercial ultraviolet diode. Absorption and emission maxima for selected donor and acceptor molecules are shown in Table 1 below.

TABLE 1

| | Absorption | Emission |
|---|---|---|
| Chromophore | Maxima (nm) | Maxima (nm) |
| Coumarin 102 | 388 | 460 |
| ATTO 390 | 390 | 479 |
| AlexaFluor 350 | 350 | 442 |
| Hoechst 33258 | 350 | 450 |
| Pyrene | 339 | 384 |

Preferred donor chromophores are coumarins. The term “coumarin” as used herein includes derivatives thereof. A preferred donor chromophore is Coumarin 102 (Cm102), and a preferred acceptor chromophore is 4-[4-(Dimethylamino)styryl]-1-docosylpyridinium bromide (Hemi22). It is thought that Cm102 associates with a nucleic acid-CTMA complex through intercalation and that Hemi22 associates through groove-binding. Other preferred donor/acceptor pairs suitable for use in the nucleic materials provided herein include Cm102 as a donor paired with fluorescein isothiocyanate (FITC) or tris-(bathophenanthroline)ruthenium (ii) chloride as an acceptor. Other suitable acceptor molecules include Eu(fod)₃, disperse red 1, sulforhodamine, (E)-2-{2-[4-(diethylamino)styryl]-4H-pyran-4-ylidene}malononitrile (DCM), or bromocresol purple (BCP) as an acceptor. Emission maxima of selected acceptors is shown in Table 2 below.

TABLE 2

| | | Emission |
|---|---|---|
| Donor | Acceptor | maxima of Acceptor |
| Coumarin 102 | Hemi22 | 600 nm |
| Coumarin 102 | FITC | ~540 nm |
| Coumarin 102 | tris-(bathophenanthroline) | ~650 nm |
| | ruthenium (ii) chloride | |

### Electrospinning

For embodiments containing fibers of the nucleic acid material, particularly when the nucleic acid material is a nucleic acid-surfactant complex, the preferred method for making the fibers is by electrospinning. Electrospinning is a well characterized technique for making nanoscale fibers and non-woven meshes from polymeric materials as described in Ner, Y., J. G. Grote, J. A. Stuart, and G. A. Sotzing, Enhanced fluorescence in electrospun dye doped DNA nanofibers. Soft Matter, 2008, 4, 1448-1453. The process of electrospinning results in extremely high surface area and porosity non-woven meshes, which permit high analyte diffusion rates. These high diffusion rates potentially improve both sensitivity and detection limits for sensor architectures.

Electrospinning provides a novel approach to processing nucleic acid surfactant (or lipid) complexes. As an example, nanofibers are prepared by electrospinning using an orthogonal arrangement of a grounded collector and a syringe containing the nucleic acid material. The nucleic acid material is electrospun into fibers that are suitable for absorbing donor and acceptor molecules or other small molecules. Alternatively, donor and acceptor molecules are introduced directly into the spin dope so that the nucleic acid material-donor and/or -acceptor matrix is formed prior to electrospinning.

Nucleic acid-material-donor/acceptor matrices have properties of enhanced emission, photostability, and small molecule interaction, and electrospinning allows these properties to be simultaneously exploited. When used with conventional polymers, such as PMMA and PVA, electrospinning distributes donor and acceptor molecules homogeneously; however, the nucleic acid material described herein provides a fixed spatial distribution of donor and acceptor molecules, formed prior to electrospinning, that both minimizes aggregation-based quenching and facilitates energy transfer.

The technique of electrospinning provides a morphology that can be exploited for both optical and sensor applications. Electrospun nanofibers amplify emission as a function of chromophore alignment and fiber geometry and provide extremely high surface area for potential analyte interactions. Other advantages of this technique include: (i) easily controlled fiber dimension and morphology; (ii) simultaneous encapsulation of multiple chromophores or other molecules of interest; and (iii) inherent scalability. The complex, regular arrangement of the nucleic acid and CTMA phases within electrospun nanofibers presents ample opportunities for the association of small molecules in discrete isolated sites.

### Film Deposition

The nucleic acid material provided herein is soluble in organic solvents. Nucleic acid material solutions are highly stable and thus, may be spin cast or dip cast. Typically, a 2% solution of a nucleic acid material, such as DNA-CTMA, in ethanol when spin cast at 2000 rpm for one minute yields films with thicknesses of 200 nm. The donor and acceptor molecules are optionally added directly to these solutions. DNA-CTMA solution consists of micelles of the CTMA encasing DNA macromolecules. These solutions also aid in dissolving organic donor and acceptor molecules.

### Properties and Applications of the Nucleic Acid Materials

Electrospun nanofibers of nucleic acid materials doped with FRET donor and acceptor molecules exhibit properties that are not easily duplicated in conventional polymer matrices. These properties include enhanced emission due to a reduction in aggregation-based quenching, an ordered distribution through interaction with the nucleic acid, and an induced alignment due to the fiber geometry. Another property of these materials is highly efficient FRET due to an ordered sequestration of donor and acceptor molecules with fixed relative orientations and separations. This property also enables higher loading of donor and acceptor molecules than otherwise possible, making higher emission intensities possible. The nanofibers also demonstrate efficient energy transfer even at very low acceptor molecule loading levels. Further, the structure of the nucleic acid material provides multiple environments for analyte interaction as a function of mesophasic morphology (e.g. nucleic acid and cationic surfactant or lipid microenvironments). Finally, these materials are also capable of rendering red-green-blue (RGB) colors through excitation with a single wavelength because the color of the emitted light can be easily controlled by varying the identity of donor-acceptor pair and the relative ratio of the dyes.

In one case, an acceptor chromophore in a FRET pair capable of absorbing donor emission and emitting in the green region of the color spectrum will render a green color. One example of such a chromophore is fluorescein isothiocyanate (FITC). Similarly, red emitting materials can be obtained from the FRET acceptor capable of emitting in the red region of the spectrum. One example of such a chromophore is Ruthenium (II) (4,7-Diphenyl-1,10-phenanthroline)₃ (Ru(DPP)₃). It is also possible to tailor color emission by rationally combining multiple chromophores. In a special case, white light emission is obtained by simultaneous emission in all of the RGB regions or in the blue and yellow regions of the color spectrum.

The nucleic acid materials described herein are suitable for multiple applications. One such application is use as white light emitting materials to replace phosphor-based coatings for diodes or fluorescent bulbs. In one embodiment, nucleic acid-based nanofibers capable of white light emission are provided. The unique, combined properties of nucleic acid and nanofiber morphology result in enhanced emission intensity of embedded chromophores.

Other applications include flat panel and flexible pixilated displays employing a variety of distributed FRET donor and acceptor pairs and sensor architectures that exploit high aspect ratio nanofibers for enhanced analyte interactions. The wide range of small molecules that interact with nucleic acids in specific modes facilitates sensor architectures. The compounds of the nucleic acid materials provided herein are also useful for probing damage to DNA or other nucleic acids, or to detect viruses, which contain nucleic acid molecules such as double-stranded RNA, into which FRET dyes could be intercalated.

Electrospun nanofibers of the nucleic acid materials described herein that are spun prior to being doped with chromophores have a variety of applications. These nanofibers can complex with FRET chromophores to form nucleic acid materials for nonradiative energy transfer, as described above. These nanofibers also have utility in detoxification applications. In detoxification applications two properties of nucleic acid nanofibers are crucial. The first is the high surface area of the nanofiber and the second is the ability of the nucleic acid to specifically bind with a wide range of molecules. Binding of nucleic acids includes intercalation, minor groove binding and surface electrostatic interactions. Examples of binding compounds include, but are not limited to, heavy metal ions, nucleic acid binding proteins, complimentary sequences, cyanine dyes, aromatic amines, nitrosamine, polymeric counter cations (e.g. chitosan), and polycyclic aromatic hydrocarbons (PAHs). PAHs are very important because they are both abundant in the environment and are carcinogenic. Heavy metal ions are of interest due to their potential presence in potable water. By combining the high surface area of nanofibers with the binding ability of nucleic acid-surfactant complexes a highly efficient filter can be fabricated.

### EXAMPLES

This specification includes descriptions of embodiments of the invention and examples of processes and materials according to the present invention. These embodiments and examples are presented only for the purpose of illustration and description and are not intended to be exhaustive or to limit the invention to the precise forms disclosed.

### Example 1

### Electrospinning of DNA-CTMA Complex

Electrospinning of an DNA-CTMA complex was carried out as follows: An orthogonal collector platform was positioned below a syringe needle assembly containing the complex. A potential was applied to the syringe needle with the collector platform as a ground. Spin dopes were produced by dissolving the DNA-CTMA complex in 200 proof ethyl alcohol for a final concentration of 10% w/w. During electrospinning, the solution was passed through a blunt tip 18G needle (ID 0.84 mm) placed at a distance of 15 cm above the collector. A constant potential of 15 kV was applied between the needle tip and the collector, and a flow rate of 0.8 ml/hr was maintained. The electrospinning was performed at ambient temperature. The spinning rate was controlled by adjusting the flow of the polymer solution using a motorized syringe pump and electrospinning was carried out for less than a minute. The electrospun fibers were collected on glass substrates placed on the grounded electrode, and dried at 60° C. in a vacuum oven for 30 minutes. As a result of this, fibers with an average fiber diameter in a range of from 250 nm to 350 nm were obtained.

### Example 2

### Crystallographic Studies

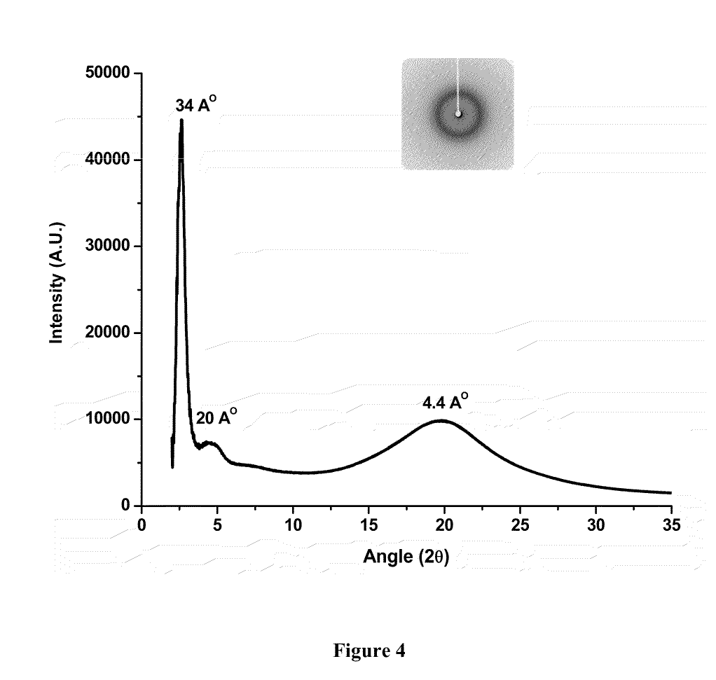

Nanofiber mesh was produced from a 10% (w/w) solution of DNA-CTMA in ethyl alcohol and chloroform in a ratio of 3:1 by weight. The nanofiber mesh was produced by electrospinning, which was carried out with an applied potential of 20 kV, a 15 cm distance between electrodes, and a flow rate of 0.8 mL/hr. FIG. 4 is an X-ray diffraction pattern of a self-standing electrospun DNA-CTMA mesh. The dried DNA-CTMA self-standing electrospun nanofiber mesh had an average fiber diameter of 300 nm. The inset of FIG. 4 shows the WAXD pattern of the nanofibers. Circular reflection peaks at 34 and 4.4 Å were observed. The electrospun fibers in the non-woven mesh adopted a completely random orientation with respect to each other. The laminar distance between DNA strands was 34 Å, a value smaller than previously reported, which implies a more compact arrangement of DNA and CTMA phases in the nanofibers.

### Example 3

### Spectroscopic Studies

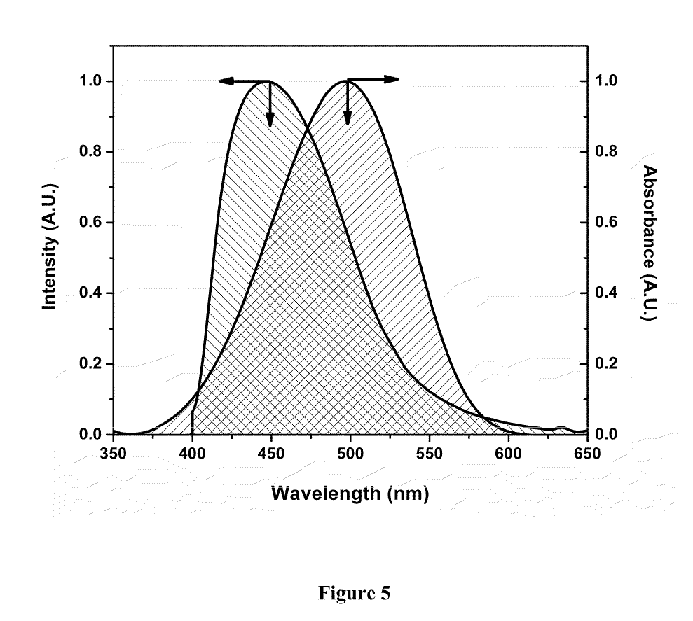

Spectroscopic studies were conducted on nanofibers of DNA-CTMA-Cm102 (donor) and DNA-CTMA-Hemi22 (acceptor), respectively. FIG. 5 is a graph showing normalized emission and UV-Visible absorption spectra of the nanofibers. The spectral overlap between the donor emission and acceptor absorption is shown in the doubly shaded region. The emission spectrum of both chromophores is red-shifted in the DNA-CTMA as compared to PMMA. The Cm102 emission maxima in PMMA is 430 nm compared to 450 nm in DNA. In the case of Hemi 22, an emission maximum in PMMA of 560 nm is observed, compared to 600 nm in DNA. This indicates that the micro-environment around the chromophore molecules is highly polar and protic, and supports association of both chromophores with the DNA phase.

### Example 4

### Fluorescence Microscopy

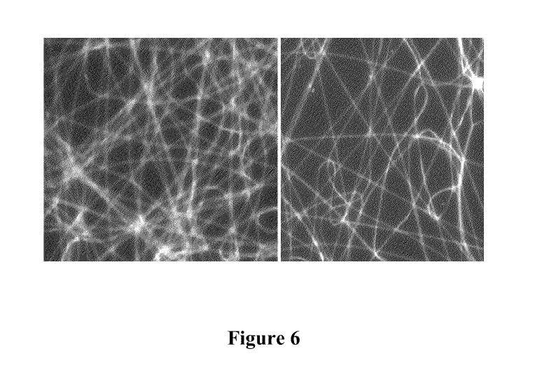

Donor doped and 1:5 acceptor:donor doped electrospun fibers were studied with fluorescence microscopy. FIGS. 6A and B are fluorescence microscopy images of excitation at 365 nm and emissions within the range of 400-700 nm. Fluorescence microscopy images clearly indicate the incorporation of the chromophore within the nanofibers.

### Example 5

### Effectiveness of Energy Transfer in DNA-CTMA Matrix

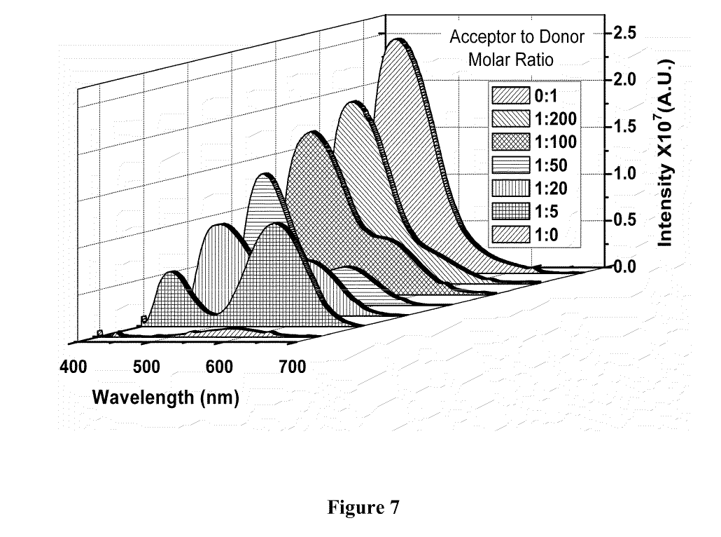

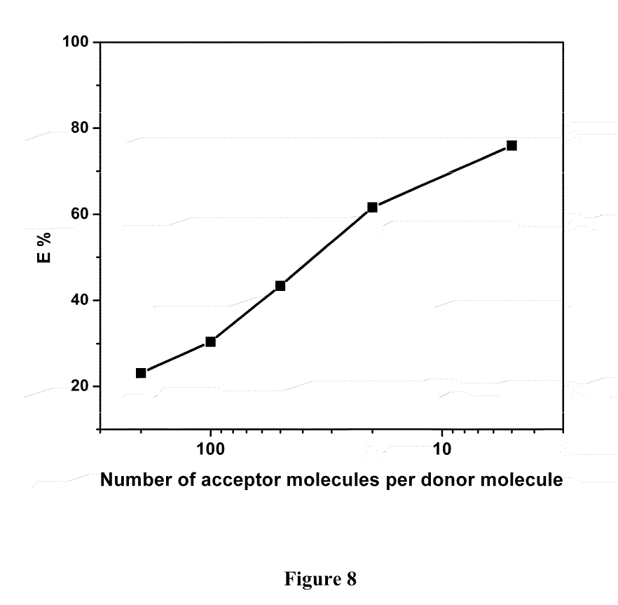

The effectiveness of the energy transfer in the DNA-CTMA matrix was studied by varying the ratio of acceptor to donor molecule. The ratio was varied between 1:200 and 1:5, and the concentration of donor dye was kept constant at 1 mole per 103 DNA base pairs to minimize self-quenching due to aggregation. FIG. 7 is a series of quenching curves for the dye doped DNA-CTMA nanofibers. In the presence of the acceptor (Hemi22), the donor (Cm102) showed quenching behavior, the magnitude of which increased at the donor emission maximum (˜450 nm) with increasing acceptor concentration. Thus, the donor emission intensity decreases as the acceptor concentration increases. The donor emission intensity decrease corresponds to an increase in acceptor intensity at ˜585 nm. The nanofiber fluorescence emission at an acceptor to donor ratio of 1:5 shows a distinct peak corresponding to acceptor emission maxima, whereas nanofibers containing only acceptor show no significant fluorescence with the same excitation wavelength. This suggests efficient FRET between the donor and acceptor chromophores within the DNA-CTMA nanofibers. FIG. 8 is a graph showing FRET efficiency plotted against acceptor to donor ratio.

### Example 6

### Tuning Color Emission

By rationally selecting a donor-acceptor pair for encapsulation and by controlling their ratio in the DNA-based material, properties of the electrospun DNA-based nanofibers can be exploited and emission very close to white light emission can be produced. At lower concentrations of the Hemi22 acceptor, the color of the fluorescence can be tuned because simultaneous emission is observed from both the acceptor and the donor.

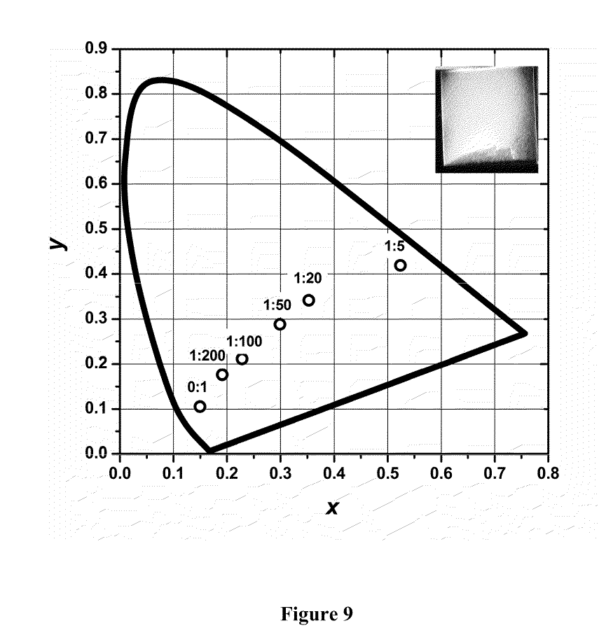

FIG. 9 is a color map for emission of DNA-CTMA-CM102-Hemi22 nanofibers with varying acceptor to donor ratios on a two dimensional projection of the CIE (Commission Internationale de E'clairage) XY chromacity diagram. With increasing acceptor concentration the color transitions from blue to orange, passing directly through pure white. The sample with acceptor to donor molar ratio 1:20 has color coordinates (0.35, 0.34) and is perceived as pure white light that has color coordinates (0.33, 0.33). The color temperature in this case was recorded to be 4650 K.

In another study, the weight ratio of dye and DNA-CTMA in nanofibers was varied from 4% to 1.33%. In this experiment, the molar ratio between the Cm102 donor and the Hemi22 acceptor was kept constant at 1:20. The changes in weight ratio also change the proximity between the donor and acceptor molecules thereby altering the FRET efficiency. The color temperature of white light emission was observed as 2909 K for 4% dye loading, 4470 K for 2% dye loading, 4650 for 1.45% dye loading and 4915 K for 1.33% dye loading. This implies that tuning of color emission is possible by changing FRET efficiency.

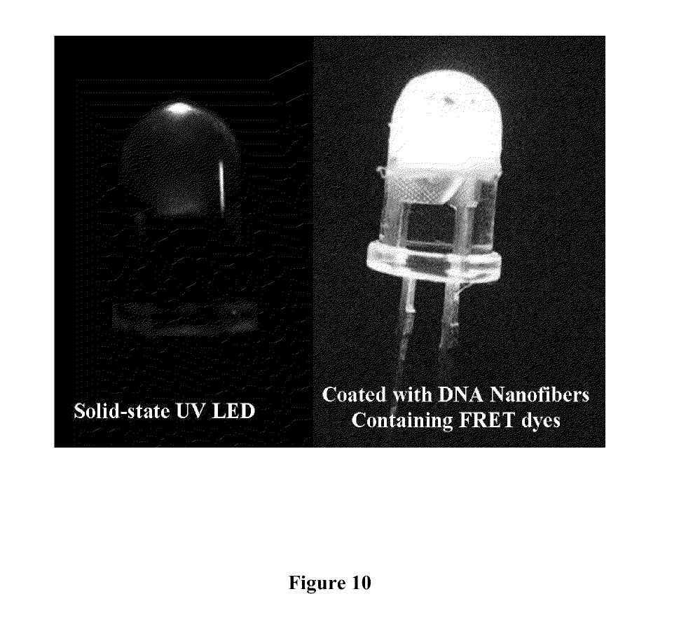

In one example, nanofibers prepared using the nucleic acid materials provided herein were deposited onto commercially available UV LEDs to convert the UV light into the full spectrum of visible light, including white light. FIG. 10 is a digital photograph of a commercially available LED, emitting at 400 nm, without (left) and with (right) FRET-based DNA nanofiber coating.

### Example 7

### Photo Stability

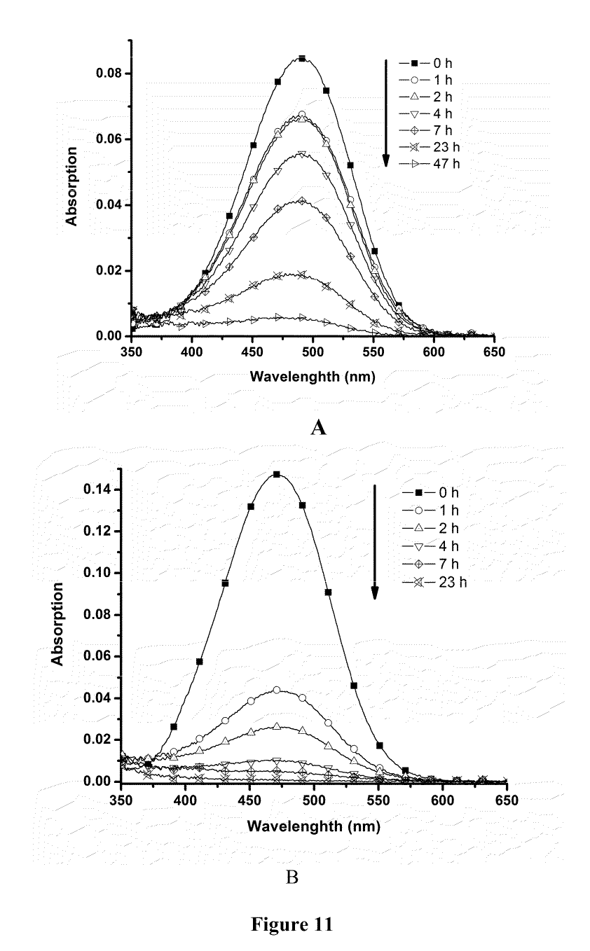

FIGS. 11A and B are graphs showing the comparative photostability of DNA and PMMA films prepared with equivalent amounts of Hemi 22 (i.e. 2.5% w/w). FIG. 11 shows the change in absorption upon exposure to UV light l=254 nm, DNA (11A) and PMMA (11). The photostability experiments were carried out by exposing film to UV light l=254 nm in a laboratory scale UV chamber. As seen in FIG. 11, the DNA films exhibited remarkable improvement in the photostability compared to PMMA films. After four hours, the PMMA films showed loss of 93% of the initial absorption while DNA based films lost 34% of the initial absorption.

### Example 8

### Triple FRET

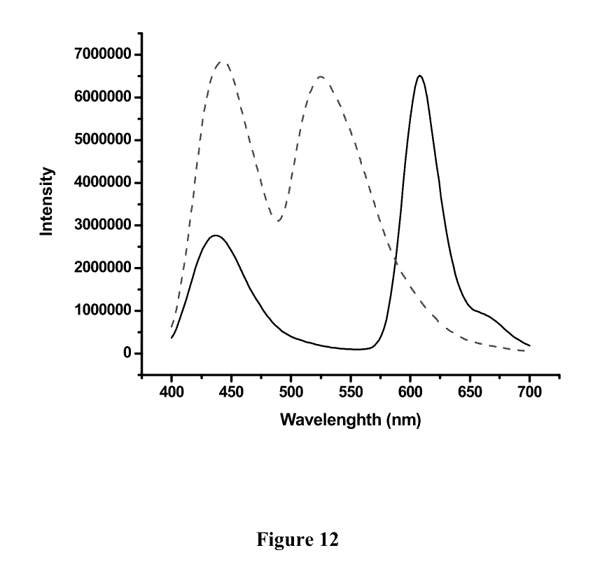

FIG. 12 is a graph showing photoluminance spectra of donor and acceptor channels formed in a DNA-CTMA films. The films were constructed as per methodology explained in the example of Spectroscopic Studies. One film contains Cm102, FITC, while the other film contains those molecules and additionally contains sulphorhodamine. Cm102 is a donor for FITC. FITC acts as an acceptor to Cm 102 and as a donor to sulphorhodamine. In the film where all three molecules are present, FITC acts as an intermediate to transfer energy from Cm102 to sulphorhodamine. The dotted line in FIG. 12 represents the photoluminance spectra of a DNA-CTMA film with only CM102 and FITC and shows peaks at about 444 nm and 528 nm representing emission of the CM102 and FITC molecules respectively. The solid line in FIG. 12 represents the photoluminance spectra of a DNA-CTMA film with Cm102, FITC, and sulphorhodamine, and shows a peak at 607 representing emission of sulphorhodamine. A peak that would correspond to emission of FITC is not observed. As a result of energy transfer the emission peak due to FITC disappeared.

### Example 9

### Sensors

DNA-CTMA nanofiber meshes with Cm102 as a donor and Ru(DPP)₃ as an acceptor were fabricated as described in prior examples herein. At acceptor to donor molar ratio 1:10, color coordinates (0.42, 0.24) were observed. The sensor architecture with these fibers was prepared by depositing these fibers onto glass slides. Ru(DPP)₃ is known to be sensitive to oxygen, and by changing the environment of these fibers it is possible to change emission of the Ru(DPP)₃ and thereby tune FRET efficiency. The color coordinates of same nanofiber mesh were observed to be (0.37, 0.21) in the 80:20 mixture of oxygen and carbon dioxide. The radiance from these fibers changed from to 5.53E-04 to 9.12E-05 watts/sr/m² in an oxygen rich atmosphere. The change in color and luminosity was significant enough to be observed by the naked eye or by any spectroscopic technique.

Preparation of a DNA-cationic surfactant complex was carried out from 500 kDa salmon DNA. Briefly, a 1% w/w aqueous solution of DNA was prepared, to which a stoichiometric amount of 1% w/w aqueous solution of CTMA was added over four hours. The resultant precipitate was washed with water and dried overnight en vacuo at 60° C. Coumarin 102 and 4-[4-(dimethylamino)styryl]-1-docosylpyridinium bromide were purchased from Sigma Aldrich and Exciton Inc, respectively.

Electrospinning was carried out with the spin dope consisting of 10% (w/w) DNA-CTMA in ethanol:chloroform (3:1, w/w). A homogeneous solution was obtained by heating at 60° C. for 30 minutes with constant stirring. Prior to electrospinning, the solution was stirred for another 5 minutes at room temperature. For dye doping, both solutions of both dyes were prepared prior to addition to DNA-CTMA. For consistency, the sequence of addition was kept as Cm102 (in ethanol) followed by Hemi22 (in chloroform). Electrospinning was performed at potential of 20 kV and the distance between the electrodes was maintained at 17 cm. The rate of spinning was controlled by adjusting flow rate using a motorized syringe pump, held constant value at 0.8 mL/hr. A stable jet between the syringe needle assembly and the collector was obtained under these conditions. Fibers were collected on the ground electrode, consisting of glass slides placed above a grounded copper plate. All experiments were carried out at room temperature and various fiber mat thicknesses were obtained by adjusting time of spinning.

Electron microscopic analysis was performed using JEOL 6335F field emission scanning electron microscope (FESEM). Fluorescence microscopy studies were performed using a Zeiss Axiovert 200M Fluorescence Microscope with a 365 nm excitation wavelength and a 400-700 nm emission window. Steady-state fluorescence measurements were performed on a Fluorolog-3 spectrofluorometer. Colorimetric measurement were performed using a PR-670 SpectraScan calorimeter under laboratory 50 W UV lamp (λ=365 nm).

It should be understood that the above examples are given only for the sake of showing that the materials and methods can be made.

Throughout this application, various publications, patents, and/or patent applications are referenced in order to more fully describe the state of the art to which these compounds and methods pertain. The disclosures of these publications, patents, and/or patent applications are herein incorporated by reference in their entireties to the same extent as if each independent publication, patent, and/or patent application was specifically and individually indicated to be incorporated by reference.

Reference is made herein to specific embodiments of the present invention. Each embodiment is provided by way of explanation of the invention, not as limitation of the invention. In fact, it will be apparent to those skilled in the art that various modifications and variations can be made in the present invention without departing from the scope or spirit of the invention. For instance, one or more features illustrated or described as part of any embodiment may be combined with or incorporated into any other embodiment to yield a further embodiment. Thus, it is intended that the present invention cover such modifications and variations as come within the scope of the appended claims and their equivalents.

The above materials and methods can be generalized to encompass a broad genus. Accordingly, the above written description is not meant to limit the invention in any way. Rather, the below claims define the invention.

What is claimed is:

1. A material for nonradiative energy transfer comprising:
(a) a nucleic acid material comprising at least one nucleic acid molecule, and
(b) a plurality of donor and acceptor molecules spaced and oriented within the nucleic acid material in an arrangement that provides nonradiative energy transfer between the donor and acceptor molecules.

2. The material of claim 1, wherein the nucleic acid material comprises a complex of the nucleic acid molecule and at least one of a cationic surfactant or a lipid with a cationic head group.

3. The material of claim 1, wherein the plurality of donor and acceptor molecules comprises at least two acceptor molecules that emit at different wavelengths.

4. The material of claim 1, wherein the donor molecules comprise coumarins, ATTO dyes, AlexaFluor dyes, Hoechst dyes, pyrenes, fluorescein isothiocyanate, or combinations thereof, and wherein the acceptor molecules comprise 4-[4-(dimethylamino)styryl]-1-docosylpyridinium bromide, fluorescein isothiocyanate, tris-(bathophenanthroline)ruthenium (ii) chloride, Eu(fod)₃, disperse red 1, sulforhodamine, (E)-2-{2-[4-(diethylamino)styryl]-4H-pyran-4-ylidene}malononitrile, bromocresol purple, or combinations thereof.

5. The material of claim 1, wherein the plurality of donor and acceptor molecules comprises at least three different molecules wherein at least one of the three molecules functions as both a donor and an acceptor.

6. The material of claim 1, wherein at least some of the donor molecules absorb ultraviolet radiation, near infrared radiation, infrared radiation, visible radiation, or combinations thereof.

7. The material of claim 1, wherein the nucleic acid material comprises a film, coating, fiber, nanofiber, or non-woven mesh.

8. The material of claim 2, wherein the cationic surfactant comprises a cationic quaternary ammonium salt.

9. The material of claim 8, wherein the cationic quaternary ammonium salt comprises cetyltrimethylammonium chloride.

10. A method of making a material for nonradiative energy transfer, the method comprising:
(a) combining a plurality of donor and acceptor molecules with a nucleic acid material, and
(b) processing the nucleic acid material to form a film, fiber, nanofiber, or non-woven mesh,
wherein the step of processing the nucleic acid material can be performed before or after the step of combining the plurality of donor and acceptor molecules with the nucleic acid material and wherein the plurality of donor and acceptor molecules are spaced and oriented within the nucleic acid material to produce the material for nonradiative energy transfer.

11. The method of claim 10, wherein the step of processing the nucleic acid comprises electrospinning, dip casting, or spin casting.

12. The method of claim 10, wherein the step of processing the nucleic acid is performed before the step of combining the plurality of donor and acceptor molecules with the nucleic acid, and wherein the step of combining the plurality of donor and acceptor molecules with the nucleic acid comprises immersing the film, fiber, nanofiber, or non-woven mesh in a solution comprising donor and acceptor molecules.

13. A material produced by the method of claim 10.

14. A method of detecting an analyte comprising:
(a) combining an analyte with the material of claim 1, and
(b) observing a change in emission characteristics of the plurality of donor and acceptor molecules.

15. The method of claim 14, wherein the change in emission characteristics comprises a color change.

16. The method of claim 14, wherein the step of observing the change in emission characteristics comprises using a spectroscopic technique.

17. A device comprising the material of claim 1, wherein the device comprises a solar cell, photovoltaic device, photodiode, sensor, flat panel display, flexible pixelated display, or fluorescent bulb.

18. The device of claim 17, wherein at least a portion of the device is covered with a thin layer of the material for nonradiative energy transfer.

19. A method for producing nonradiative energy transfer comprising:
(a) irradiating a material comprising a nucleic acid material and a plurality of donor and acceptor molecules, wherein the plurality of donor and acceptor molecules are spaced and oriented within the nucleic acid material in an arrangement that provides nonradiative energy transfer between the chromophores; wherein the irradiation places at least one donor chromophore into an excited state;
(b) transferring energy from the at least one donor molecule in an excited state to at least one acceptor molecule.

20. The method of claim 19, wherein with the irradiation comprises ultraviolet radiation, near infrared radiation, infrared radiation, visible radiation, or combinations thereof.

21. The method of claim 19, wherein transferring energy from the donor molecule to the acceptor molecule comprises Förster Resonance Energy Transfer, production of visible light or production of near infrared luminescence.

22. A composition comprising a combination of a plurality of the materials for nonradiative energy transfer of claim 1, wherein the combination produces a predetermined emission wavelength.
